(19) United States
(12) Patent Application Publication
(10) Pub. No.: US 2026/0148847 A1
(43) Pub. Date: May 28, 2026

(54) OPHTHALOMOSCOPE APPLICATION

(71) Applicant: Abu Dhabi University, Abu Dhabi (AE)

(72) Inventors: Taimur Hassan, Abu Dhabi (AE); Basma Bashir, Abu Dhabi (AE); Muhammad Usman Akram, Islamabad (PK); Irfan Hussain, Abu Dhabi (AE); Jawad Yousaf, Abu Dhabi (AE); Mohammed Ghazal, Abu Dhabi (AE)

(73) Assignee: Abu Dhabi University, Abu Dhabi (AE)

(21) Appl. No.: 18/957,742

(22) Filed: Nov. 23, 2024

(51) Int. Cl. G16H 50/20; A61B 3/12; G16H 15/00

(52) U.S. Cl. CPC G16H 50/20; A61B 3/12; G16H 15/00

(57) ABSTRACT

A method includes receiving an electronic fundus scan. The method also includes determining retinal disease information based on receiving the electronic fundus scan.

### BACKGROUND

Retinopathy refers to a group of retinal diseases that can cause severe visual impairments or even blindness if left untreated. Currently, computer-aided screening systems are used in clinical practice to identify retinal lesions from the retinal imagery for screening retinal diseases. However, such systems have inherently two main limitations. First, current systems they require expensive machinery, and current systems require a level of computational power which is not available within portable handheld devices. As such, the current systems generally only available at medical facilities and cannot be used on a portable handheld device.

### BRIEF DESCRIPTION OF DRAWINGS

FIG. 1 is a diagram of an example system;

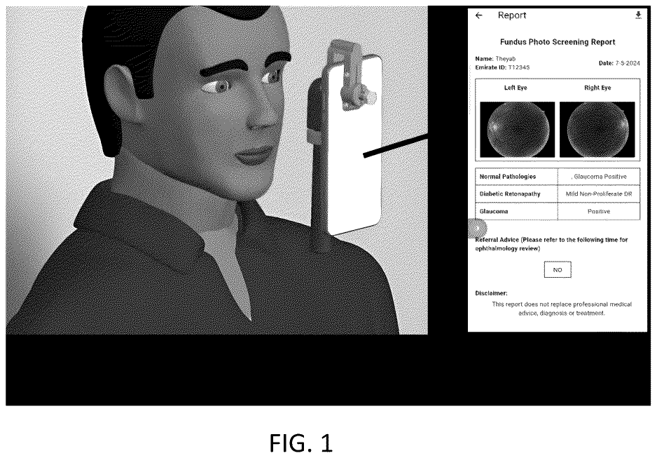

FIG. 2 is a diagram of an example electronic screen associated with an example system described herein;

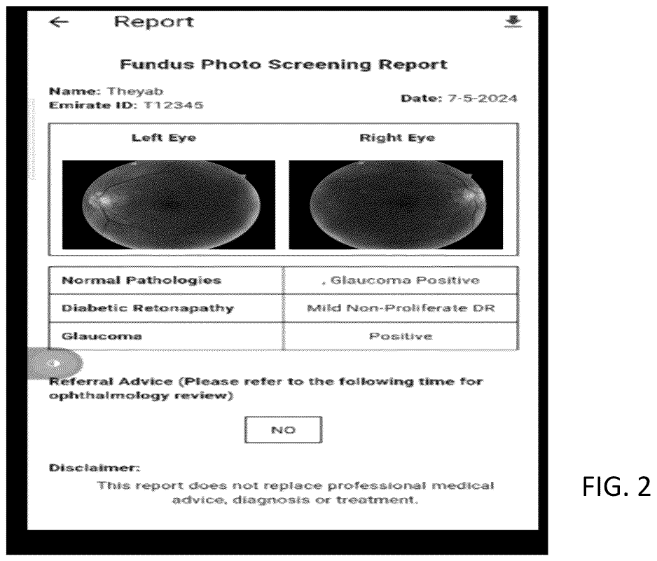

FIG. 3 is a diagram of an example system;

FIG. 4 is a diagram of an example electronic screen;

FIG. 5 is a diagram of an electronic screen;

FIG. 6 is a diagram of an example screen;

FIG. 7 is a diagram of an example system;

FIG. 8 is a diagram of an example system;

FIG. 9 is a diagram of an example flowchart;

FIG. 10 is a diagram of an example graphical image;

FIG. 11 is a diagram of an example graphical image;

FIG. 12 is a diagram an example graphical image;

FIG. 13 is a diagram of a process within an example system;

FIG. 14 is a diagram of another process within an example system;

FIG. 15 is a diagram of another process within an example system;

FIG. 16 is a diagram of another process within an example system;

FIG. 17 is a diagram of another process within an example system;

FIG. 18 is a diagram of another process within an example system;

FIG. 19 is a diagram of another process within an example system;

FIG. 20 is a diagram of an example table;

FIG. 21 is a diagram of an example graphical image;

FIG. 22 is a diagram of an example graphical image;

FIG. 23 is a diagram of an example table;

FIG. 24 is a diagram of an example table;

FIG. 25 is a diagram of an example computing networking system; and

FIG. 26 is a diagram of an example computer.

### DETAILED DESCRIPTION OF PREFERRED EMBODIMENTS

The following detailed description refers to the accompanying drawings. The same reference numbers in different drawings may identify the same or similar elements.

Systems, devices, and/or methods described herein are a cost-effective, portable, and AI-enabled system that allows for a hand-held user device (such as a smartphone) to be used as a ophthalmoscope. In embodiments, the user device includes an AI (artificial intelligence)-enabled smartphone application (exam application) in which patients can register themselves in order to acquire retinal fundus scans and automatically analyze them to screen different retinal diseases.

In embodiments, clinical reports can then be generated from the exam application, which includes patient demographic information as well as AI screening results. In embodiments, the electronic reports can then be shared with doctors and hospitals to develop further treatment plans. In embodiments, the autonomous screening of retinal diseases within the exam application can be performed by any deep learning model, including a convolutional transformer architecture, embedded within the exam application. In embodiments, the convolutional transformer architecture extracts electronic information about retinal lesions from acquired retinal fundus scans that are generated from electronic imagery received via the user device camera. In embodiments, the exam application uses the lesion electronic information to then diagnose different retinal diseases. As described herein, a AI module within the exam application can achieve a high correlation coefficient with the grading of the expert clinicians toward screening retinal diseases, such as diabetic retinopathy and glaucoma.

Accordingly, the systems, methods, and/or devices described herein provide for an AI-enabled patient registration and screening application that is cost-effective, portable, and user-friendly invention. Furthermore, the systems, methods, and/or devices described herein possess AI capabilities to robustly screen retinal diseases. In embodiments, the screening results are formulated automatically in clinical reports which can be shared with the doctors and clinicians through the exam application. Similarly, the exam application can aid the patients to self-diagnose and monitor their retinal health regularly and possibly prevent the sudden vision loss.

In embodiment, a user device attachment allows the user to acquire fundus scans (via the exam application) and analyze them to generate screening reports as per a clinical standard. In embodiments, the exam application and the smartphone attachment can be used with any smartphone or any other type of portable electronic device. Additionally, the exam application can register the patients, control electronic communications and processes of the user device to acquire retinal fundus images, screen these images and generate clinical reports. In embodiments, the exam application can store these reports in a server (such as a cloud server) where these reports can later be shared with the doctors and hospitals. In embodiments, the exam application can also include a deep learning model, particularly the convolutional transformer architecture, within the smartphone application to screen retinal diseases, such as DR and glaucoma.

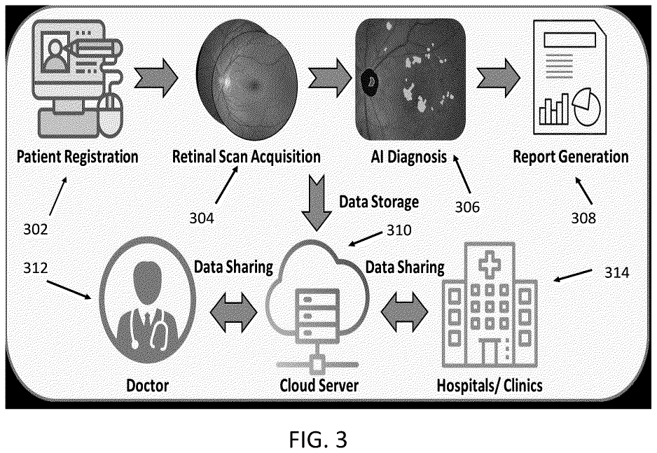

FIG. 3 is an example block diagram describing an example process associated with the exam application. At block 302, users (e.g., patients) use the exam application create their electronic accounts in the exam application. At this step, the user creates a username, password, and other logistic information so as to be able to use the exam application to record the scans from the link, generate screening reports, save them in the phone local storage or over the cloud in order to share it with the doctors and hospitals. In embodiments, exam application uses these electronic accounts to allow complete management of a patient's history, checkup, screening results, follow-ups, recommendation from doctors, and other types of electronic communication.

After creating and logging into the exam application, at block 304, the patients (or a person assisting the patient) can use the exam application (along with the user devices) to take real-time fundus scans of the patient's left and right eye. At 306, these scans (e.g., images) are then passed to the convolutional transformer architecture which screens the acquired scans against retinal diseases, such as DR and glaucoma. At 308, the exam application generates screening reports that can be shared with the hospitals and registered doctors for the follow-up and possible treatments.

At 310, retina imagery may be stored in a database such as a cloud storage database. At 312, such retina imagery (as well as other electronic information) may be sent to doctors; and at 314, such retina imagery (as well as other electronic information) may be sent to medical centers and hospitals.

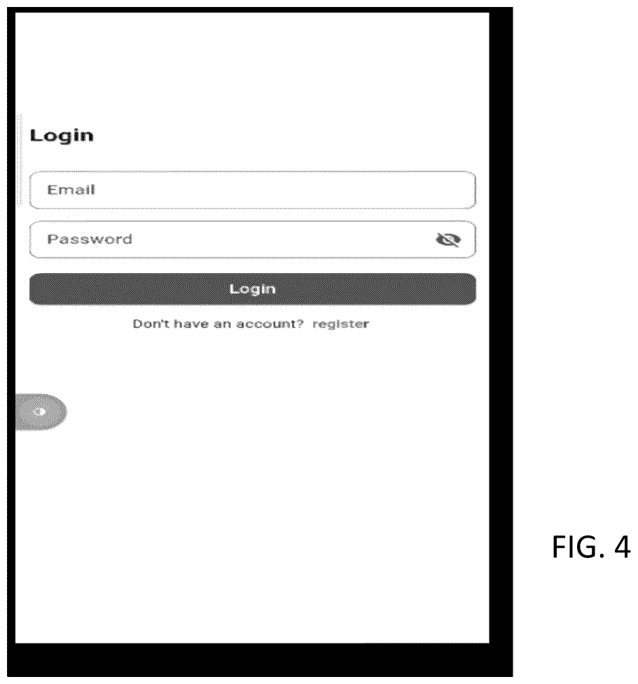

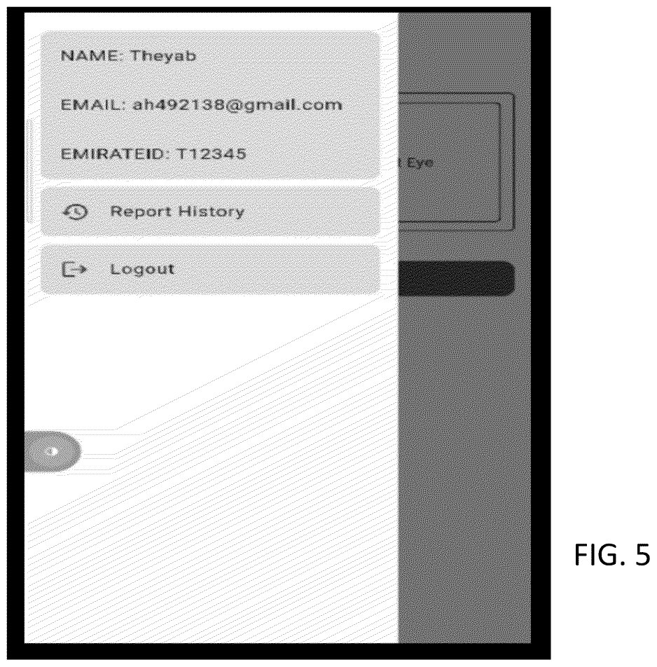

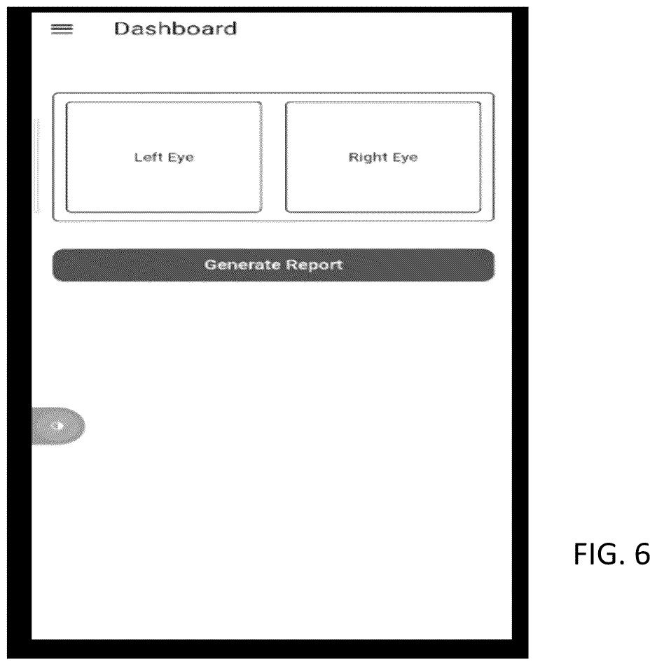

FIGS. 4-6 describe electronic displays generated by an exam application. FIG. 4 is an example electronic display for signing up and logging in. FIG. 5 is an example electronic display that describes an electronic dashboard with various information. FIG. 6 is an example electronic display that provides a user to then use the exam application to take retinal scans.

Once the registration and log-in is completed, the exam application can be used (with the user device camera) to receive retinal fundus images. In embodiments, these acquired fundus images are then stored in a database against the logged-in user and they can be passed to the AI systems within the exam application for screening purposes. Furthermore, to allow a user to use the user device and exam application by themselves to acquire the retinal scan imagery, a custom-designed attachment which can be used with any smartphone. Accordingly, the retinal scan imagery can be acquired using the smartphone camera operated through the exam application. In embodiments, the exam application may be considered as a mydriatic process, which means that the user may need to undergo a pupil dilation process before the scans can be acquired and are passed to the deep learning model for screening purposes.

In embodiments, after acquiring the left and right eye fundus scan imagery of the person using the exam application, the scans are stored in the database against the signed-in patient record. Afterward, the scans can be passed to the convolutional transformer system which analyzes the retinal diseases from the acquired fundus scans. In embodiments, the convolutional transformer system takes the acquired scans (in some instances offline) and determines whether there are any lesion-aware screening of retinal diseases.

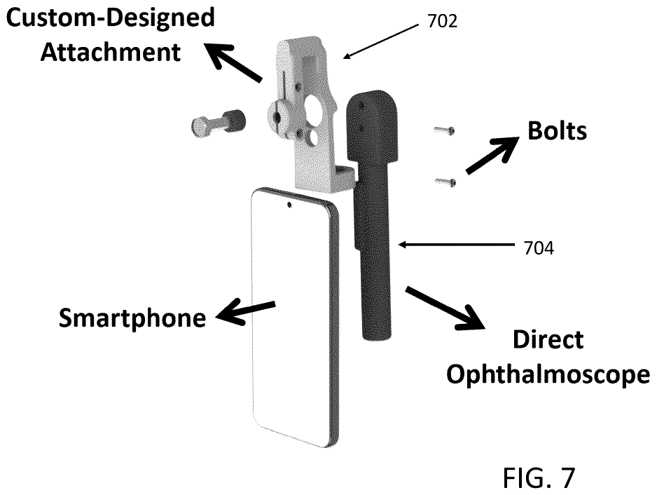

FIG. 7 describes an example attachment with parts 702 and 704.

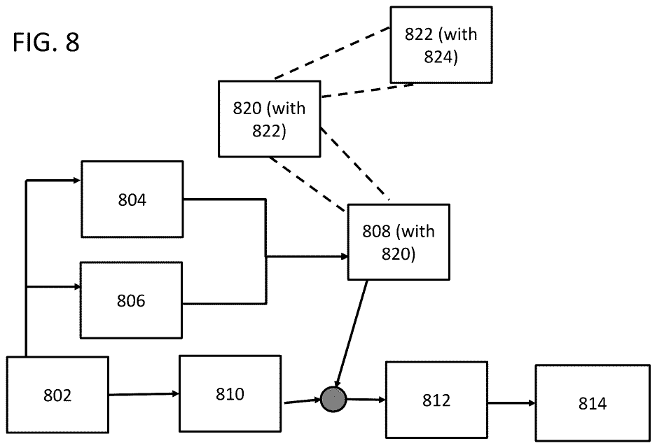

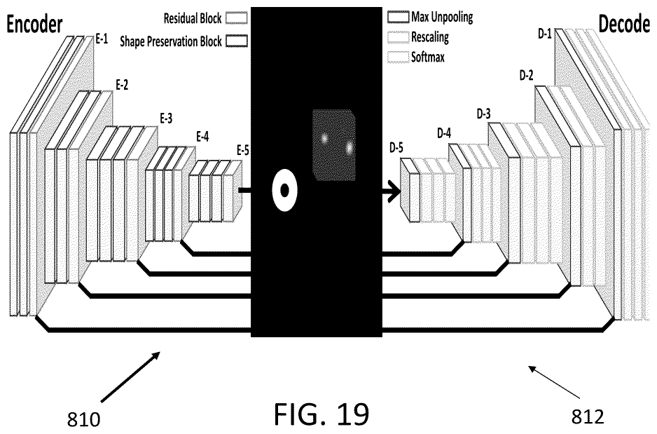

FIG. 8 describes a convolutional transformer system 800. In embodiments, convolutional transformer system 800 can receive electronic fundus scans as an input and screen different retinal diseases from them, such as DR and glaucoma. In embodiments, a further description of the convolutional transformer system is shown in FIG. 8. The architectural description of the convolutional transformer system is also described shown in FIG. 19. In embodiments, the convolutional transformer system consists of three parts: encoder 810, transformer 808, and decoder 812. In embodiments, input scan 802 is first passed to encoder 810 which generates the latent feature representations to distinguish different retinal pathologies. Moreover, the scan is also decomposed into non-overlapping sequenced patches 806 from which the latent and the flattened projections are generated.

In embodiments, the flattened projections are obtained through the positional embeddings 804 of the sequenced patches, whereas the latent projections are generated using linear embeddings from patch decomposition 806. In embodiments, these projections are then added together and are passed to the t transformer encoders (t=3) of transformer 808, which compute the contextual multi-head self-attentional distributions (dubbed as “pt”).

In embodiments, these feature distributions are concatenated with the latent space representations of the encoder block (fe) output of encoder 810, and the combined distribution (fd) (which is of the output of transformer 808 and the output of encoder 810) is passed to decoder 812, which extracts the retinal lesions through rescaling blocks. In embodiments, the detailed description of encoder 810, transformer 808, and decoder block 812 within the convolutional transformer system is further described.

In embodiments, encoder 810 (which are made up of encoder blocks) within the convolutional transformer system is responsible for generating the latent features distribution fe(x) from the input scans x of size Rw×Cc×Cl, where Rw represents the rows, Cc represents the columns, and Cl represents the channels. In embodiments, the encoder consists of five levels (E-1 to E-5), where each level contains three to four shape preservation and residual blocks. In embodiments, the encoder blocks allow encoder 810 to produce accurate representation of the retinal lesions in order to yield distinct feature maps.

In embodiments, to further boost the separation of latent features associated with different retinal lesions, fe is convolved with the transformer projections pt (the output of transformer 808, that yield the fused feature representations fd=fe*pt in which the similar features between fe and pt are amplified and the heterogeneous representations are suppressed. In embodiments, the fused features are passed to the decoder 812 to extract the retinal lesions provided an output image that may shows possible lesions associated with retinal diseases, such as DR and glaucoma.

In embodiments, transformer block within transformer 808 consists of t encoders, where t is empirically determined to be 3, i.e., t=3, yielding, T-1, T-2, and T-3 encoders. In embodiments, these encoders are coupled together in a sequential fashion to produce pₜ. Here, the input retinal scan x is first chunked-down into non-overlapping squared patches x^{p}∈R^{P×P×C}^{ₗ}, where P represents the x^{p} resolution, such that

$$P = \sqrt{\frac{{RC}_{h}}{{n}_{p}}} ,$$

and nₚ represents the number of patches. Afterward, we generate the positional embeddings

$${x}_{i}^{e}$$

corresponding to patch

$${x}_{i}^{p} ,$$

i.e., x^{e}∈R^{P×P×C}^{ₕ}, from which the flatten projections are computed, i.e.,

$${f}_{p} ( {x}_{i}^{e} ) .$$

Similarly, we obtain the linear projection

$${l}_{t} ( {x}_{i}^{p} )$$

for the patch

$${x}_{i}^{p} ,$$

and then we resize

$${f}_{p} ( {x}_{i}^{e} ) and {l}_{t} ( {x}_{i}^{p} )$$

to l dimensions and generate the sequenced embeddings (for

$${x}_{i}^{p}$$

patch), i.e., qᵢ, through

$$\$ {q}_{i} = {l}_{t} ( {x}_{i}^{p} ) + {f}_{p} ( {x}_{i}^{e} ) .$$

Repeating this process for all nₚ patches produce the combined projections q^{o}:

$${q}^{o} = [ {l}_{t} ( {x}_{0}^{p} ) ; {l}_{t} ( {x}_{1}^{p} ) ; … ; {l}_{t} ( {x}_{{n}_{p} - 1}^{p} ) ] + [ {f}_{p} ( {x}_{0}^{e} ) ; {f}_{p} ( {x}_{1}^{e} ) ; … ; {f}_{p} ( {x}_{{n}_{p} - 1}^{p} ) ] , \qquad ( 1 )$$

$${q}^{o} = [ {q}_{0} ; {q}_{1} ; … ; {q}_{{n}_{p} - 1} ] , \qquad ( 2 )$$

q^{o} is then forwarded to the transformer encoder, at head j. Afterward,

$${q}_{j}^{o}$$

will be normalized to produced

$${q}_{j}^{′ o} .$$

### Then,

$${q}_{j}^{′ o}$$

will be linearly decomposed into query (Qⱼ), key (Kⱼ), and value (Vⱼ) pairs via learnable weights, such that,

$${Q}_{j} = {q}_{j}^{′ o} {w}_{q} , K = {q}_{j}^{′ o} {w}_{k} , and V = {q}_{j}^{′ o} {w}_{v} .$$

The contextual self-attention at head j will be computed as:

$${A}_{j} ( {q}_{j}^{′ o} ; {Q}_{j} , {K}_{j} , {V}_{j} ) = σ ( \frac{{Q}_{j} {K}_{j}^{T}}{\sqrt{l}} ) {V}_{j} , \qquad ( 3 )$$

where σ(⋅) denotes the softmax function. Moreover, the contextual self-attention maps from multiple heads will then be fused to generate multi-head self-attention distribution (φ_{CMSA}(q′^{o})), as shown below:

$${φ}_{C M S A} ( {q}^{′ o} ) = [ {A}_{0} ( {q}_{j}^{′ 0} ; {Q}_{0} , {K}_{0} , {V}_{0} ) ; {A}_{1} ( {q}_{j}^{′ 1} ; {Q}_{1} , {K}_{1} , {V}_{1} ) ; … ; {A}_{h - 1} ( {q}_{j}^{′ h - 1} ; {Q}_{h - 1} , {K}_{h - 1} , {V}_{h - 1} ) ] . \qquad ( 4 )$$

Apart from this, φ_{CMSA}(q′^{o}) will also be added with q^{o}, and their normalized representations will be forwarded to the feed-forward block to produce the projections of the first transformer encoder:

$${P}_{T 1} = {∅}_{f} ( {( {φ}_{CMSA} ( {q}^{′ o} ) + {q}^{o} )}^{′} ) + {( {φ}_{CMSA} ( {q}^{′ o} ) + {q}^{o} )}^{′} , \qquad ( 5 )$$

where Ø_{f}(⋅) denotes the learnable feed-forward function. After computing p_{T1}, it will be passed to the second transformer encoder, that will produce p_{T2} in a similar fashion, and p_{T2} will be passed to the third transformer encoder which will produce p_{T3}. For the third cascaded transformer encoder, the projections pₜ will be equal to pₜ, i.e., pₜ=p_{T3}. Afterward, pₜ will be fused with the fₑ to generate f_{d}, and f_{d} is passed to the decoder block to extract the retinal lesions.

We convolve fₑ with pₜ to obtain f_{d} which is then passed to decoder 812 for robustly extracting the retinal lesions and displayed at 814. In embodiments, decoder 812 has decoder blocks that consists of five levels where each level contains one max unpooling and two to three rescaling blocks. Moreover, the skip-connections are also established between encoder and decoder block to overcome the degradation problem of the model during retinal lesions segmentation. Also, decoder 812 has softmax layer at the head to classify each pixel within the candidate scan into one of the retinal lesions' categories.

In embodiments, after extracting the retinal lesions from the candidate fundus scan using convolutional transformer model 800, the extracted information about retinal lesions can be used towards screening the retinal diseases (e.g., such as diabetic retinopathy, glaucoma, etc.).

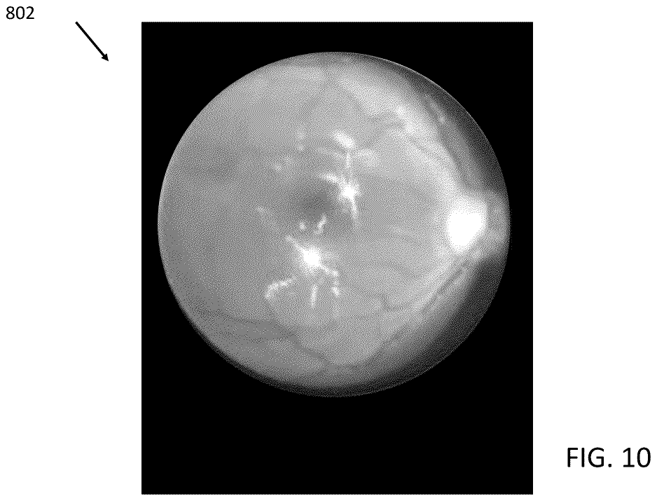

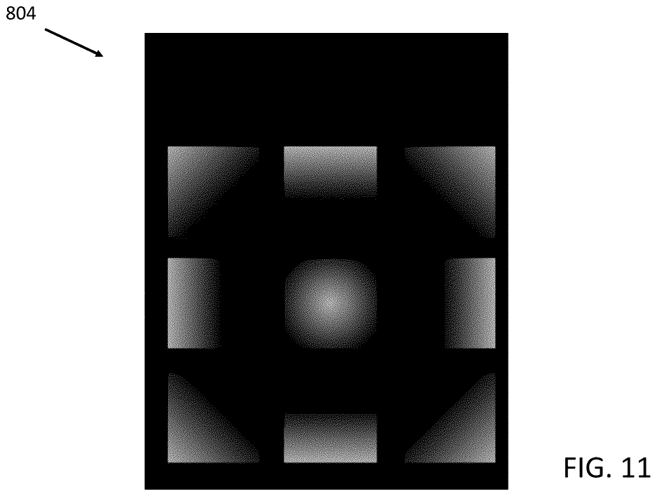

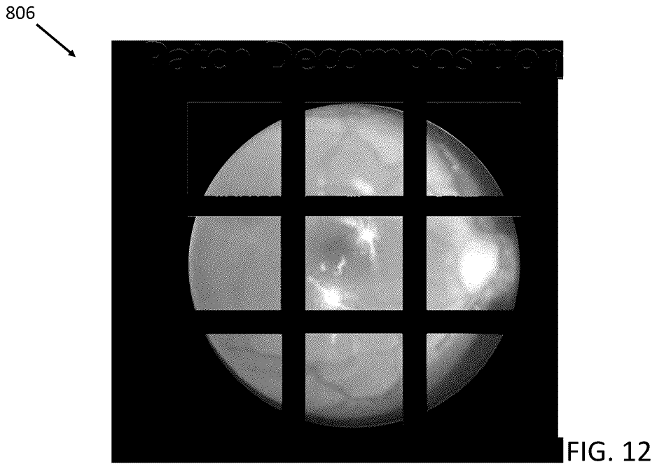

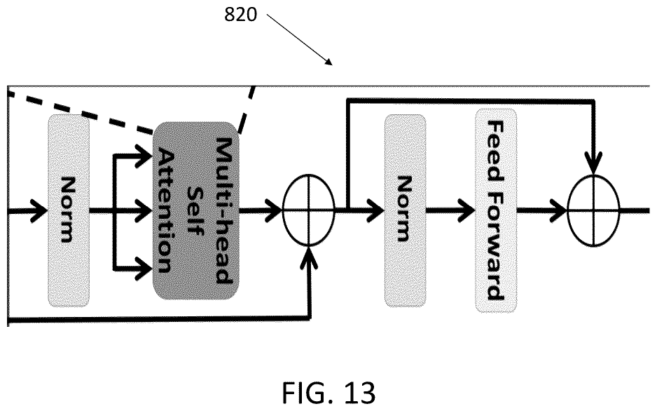

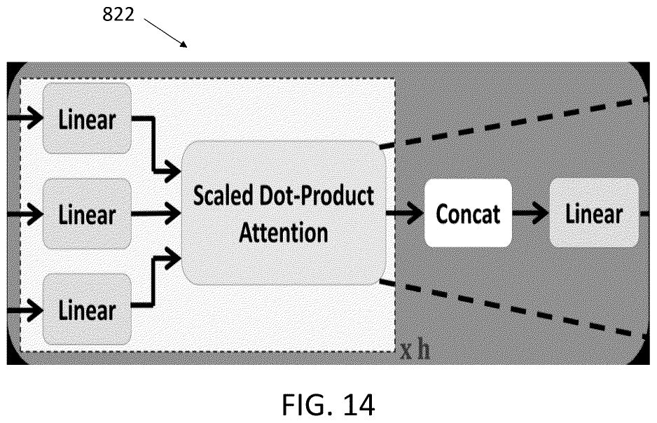

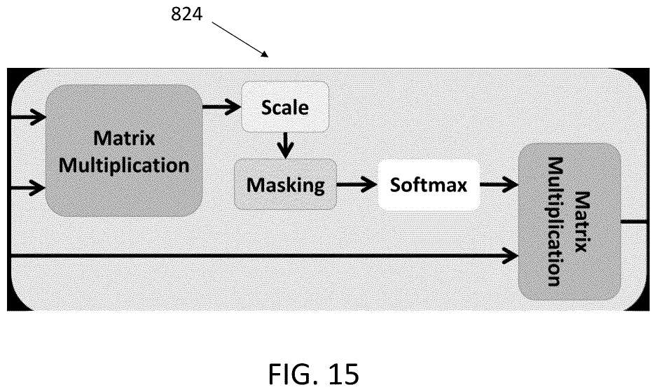

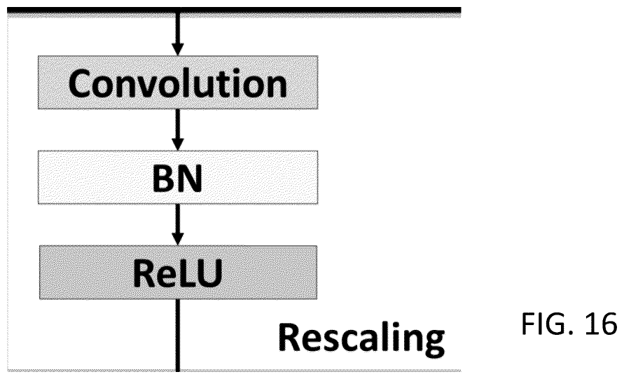

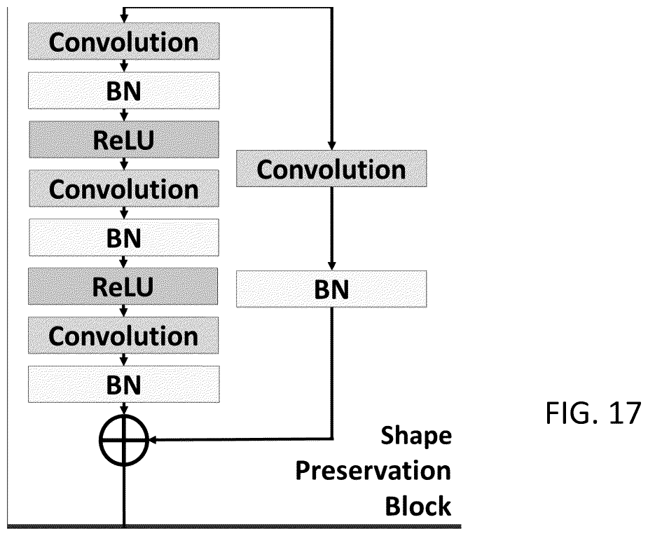

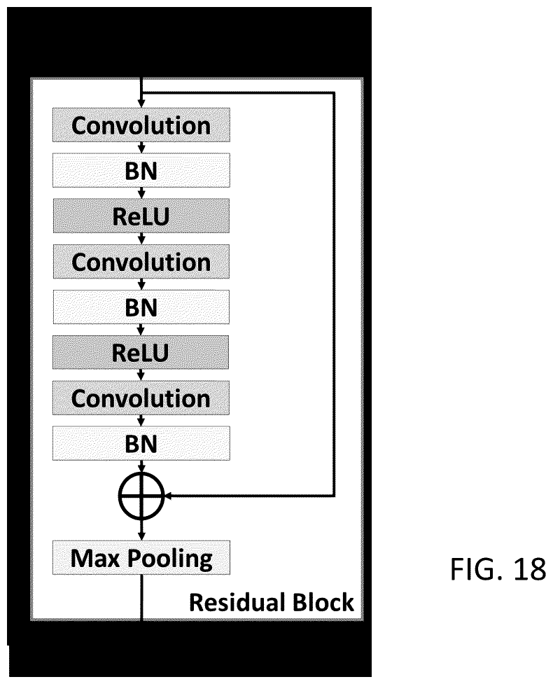

FIGS. 10 to 15 and 19 further describe the elements of FIG. 8. FIG. 10 describes insert scan 802. FIG. 11 describes positional embeddings 804. FIG. 12 describes patch decomposition 806. FIG. 13 describes the vision transformer's encoder 820. FIG. 14 describes the vision transformer's multi-head self-attention 822. FIG. 15 describes the vision transformer's scaled dot-product attention 824. FIGS. 16 to 18 describe the Rescaling, Shape Preservation, and Residual blocks, respectively, which are employed within the convolutional transformer model.

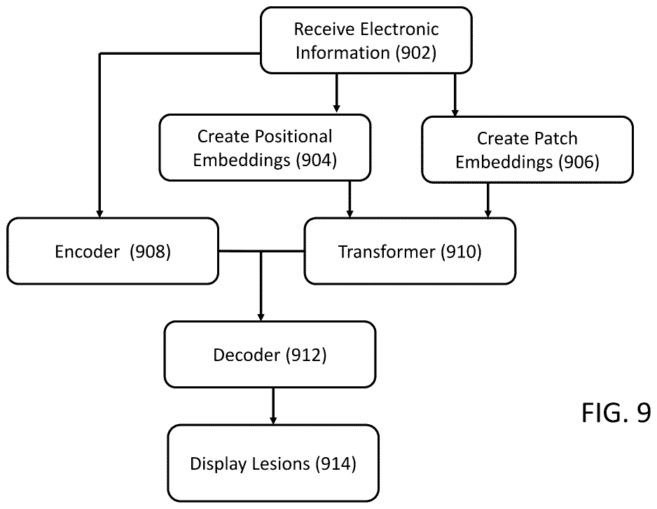

FIG. 9 is an example flowchart 900 that describes the process of determining any existence of one or more eye diseases. At step 902, electronic information is received about a person's eye. In embodiment, the electronic information may be imagery of a portion, or all, of a person's eye. At step 904, positional embeddings are generated which are then used. At step 906, a patch decomposition is conducted. At step 908, electronic information is sent to an encoder. At step 910, the combination of the output of steps 904 and 906 are sent to a transformer (e.g., transformer 808). As shown in FIG. 9, the output of step 910 is sent, at step 912, to a decoder. Furthermore, the electronic information at step 902 is also sent to the decoder. In embodiments, the decoder (e.g., decoder 912) generates electronic imagery that describes any lesions within the person's eye (for which electronic information was sent at step 902)

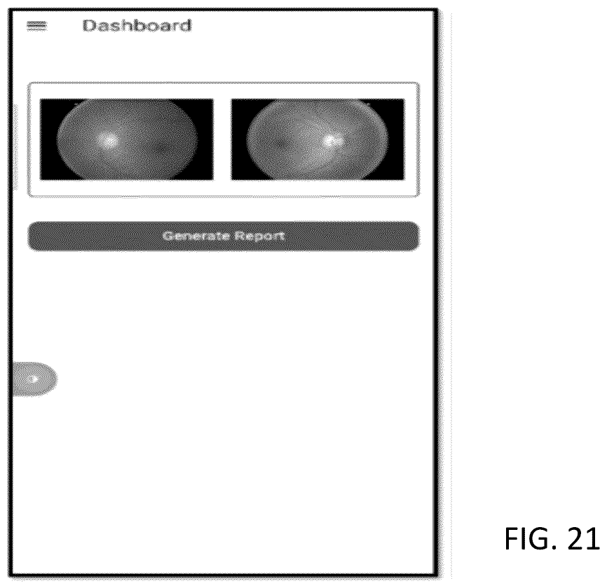

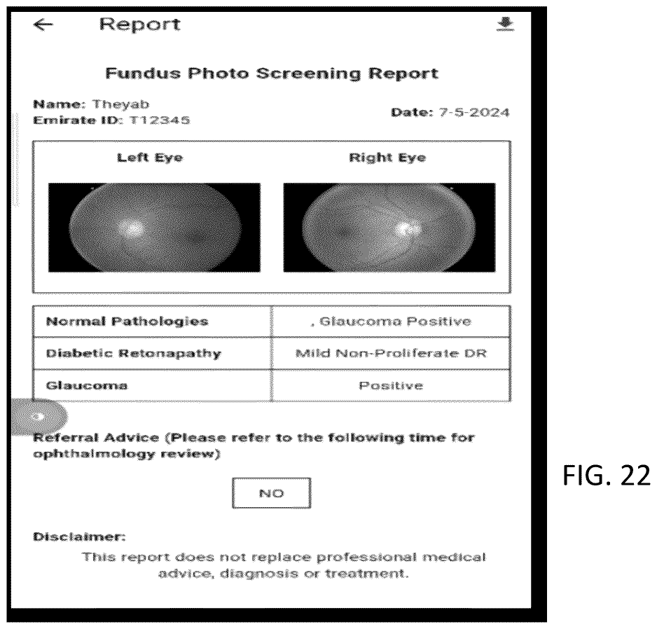

In embodiments, once the presence of retinal diseases are detected by the convolutional transformer model, the exam application can automatically generate the screening reports in which the AI results are embedded along with the patient demographic information. The visual examples showcasing the generated reports by the exam application can be seen in FIGS. 21 and 22. In embodiments, the electronic reports contain demographic information about the patients, as well as the acquired scans/images, and the screening results generated by the convolutional transformer system. Within these reports, a disclaimer may be added that these reports cannot replace ophthalmologists and clinician's opinion, and treatment recommendations.

In embodiments, the exam application can electronically communicate any electronically generated report to other computing/storage devices so that the information can be shared with doctors and medical facilities. In embodiments, the reports can be exported in a PDF format which can be stored in the cloud storage and can be shared with the doctors for further analysis or for getting the treatment plan.

In embodiments, the exam application can be trained using electronic data. For example, an electronic dataset can contain 255 fundus scans, from which 43 represent healthy pathology, 138 had DR symptoms, and 74 were affected by glaucoma. In other example, electronic datasets with different information can be provided to the exam application. In this non-limiting example, the scans are acquired after dilating the pupils, and the dataset has been electronically marked by the panel of expert clinicians, both at the pixel level and scan level, respectively, for extracting the retinal lesions and diagnosing the retinal diseases, such as DR and glaucoma. Apart from this, we used 80% of the scans for and the rest for testing purposes.

The convolutional transformer model within RetMobile is trained for 20 epochs on the RetMobile training dataset, where each epoch consisted of 512 iterations. The optimizer used during the training was ADADELTA (however other optimizers may be used) having the default learning and decay rate of 1.00 and 0.95, respectively. Moreover, in each iteration, we used the dice entropy loss function to constrain the convolutional transformer model (as expressed in Eq. 6).

$${L}_{d e} = {α}_{1} {L}_{d} + {α}_{2} {L}_{e} \qquad ( 6 )$$

$${L}_{d} = \frac{1}{N} {\sum }_{i = 1}^{N} ( 1 - \frac{2 {\sum }_{j = 1}^{C} {t}_{i , j} {p}_{i , j}}{{\sum }_{j = 1}^{C} {t}_{i , j}^{2} + {\sum }_{j = 1}^{C} {p}_{i , j}^{2}} ) \qquad ( 7 )$$

$${L}_{e} = - \frac{1}{N} {\sum }_{i = 1}^{N} {\sum }_{j = 1}^{C} {t}_{i , j} log ( {p}_{i , j} ) \qquad ( 8 )$$

where α_{1,2} represent the loss functions weights, Lₑ denotes the categorical cross-entropy loss, L_{d} represent the dice loss, p_{i,j} reflect the predicted probability for the i^{th} sample and j^{th} class, t_{i,j} denotes the true labels of the i^{th} sample for the j^{th} class, C is the total number of disease categories, and N denotes the batch size. Moreover, the model is trained on Lambda Labs Tensorbook Intel Core i7-9750H@2.6 GHz, 32 GB RAM, and a single NVIDIA RTX 2080 Max-Q GPU with cuDNN v7.5 and a CUDA Toolkit 10.1.243.

To evaluate the classification performance, metrics derived from a confusion matrix (a matrix that describe the correctly classified and incorrectly classified samples belonging to healthy, glaucoma and DR classes), such as success rate (accuracy), positive predicted value (PPV), true positive rate (TPR), and the F1 scores. Moreover, the computational size of the deep learning models is measured using this number of parameters.

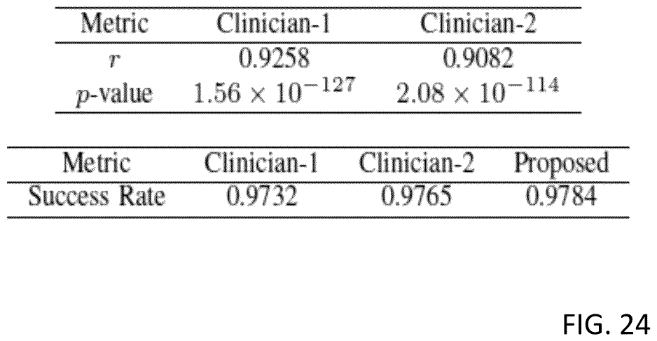

In embodiments, the screening performance of the RetMobile device is also compared with the grading of the clinicians on the same scans. This comparison allowed to determine the degree of alignment between the screening performance of the RetMobile device and the recommendations of the clinicians. In a non-limiting example, the total number of fundus scans can be 300, where 105 of the scans were acquired from the glaucomatous subjects, 135 scans were acquired from the DR subjects, and 60 scans were acquired from the healthy subjects. As shown in FIG. 24, the exam application achieved the success rate of 0.9784 for correctly recognizing healthy, DR and glaucoma-affected scans. Moreover, the screening performance of the exam application matches with the grading of the clinicians where it achieved the statistically significant correlation coefficient of 0.9732 with the first clinician (p<0.05), and a statistically significant correlation coefficient of 0.9765 with the second clinician (p<0.05). Accordingly, the exam application can be used in the real-world for screening retinal diseases, such as DR and glaucoma.

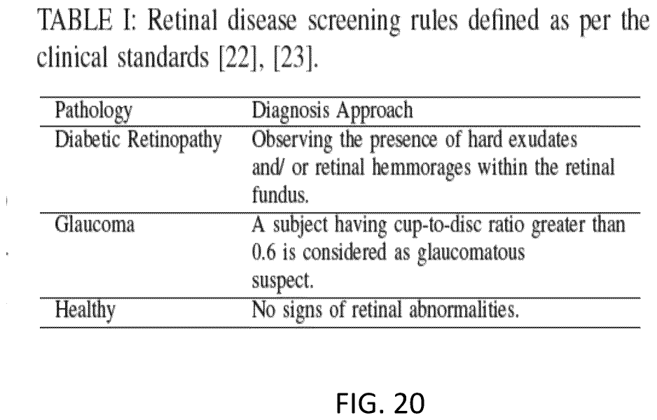

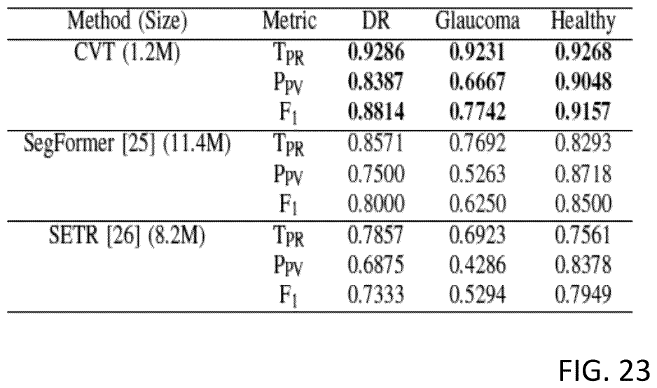

As shown in FIG. 23, the exam application can be coupled with any deep learning model for screening retinal diseases, such as DR and glaucoma. As shown in FIG. 23, the convolutional transformer system of the exam application outperforms other methods by 9.23% for screening DR, 19.27% for screening glaucoma, and 7.17% for screening healthy pathologies, in terms of F1 scores, respectively. Moreover, in terms of computational size, compared to other models. The improvements achieved by the convolutional transformer model directly relate to its capacity toward paying attention to retinal lesions (abnormalities) which enables accurate recognition of retinal diseases as per the clinical standards (shown in FIG. 20). the convolutional transformer model provides improved/better results

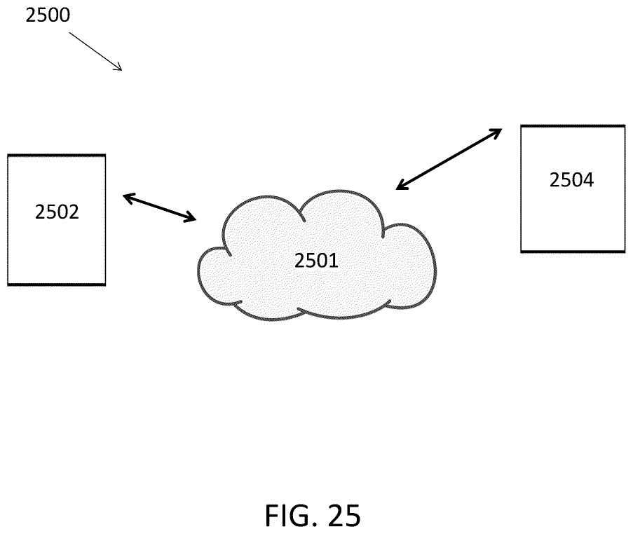

FIG. 25 is a diagram of example environment 2500 in which systems, devices, and/or methods described herein may be implemented. FIG. 5 shows network 2501, user device 2502, and user device 2504.

Network 2501 may include a local area network (LAN), wide area network (WAN), a metropolitan network (MAN), a telephone network (e.g., the Public Switched Telephone Network (PSTN)), a Wireless Local Area Networking (WLAN), a WiFi, a hotspot, a Light fidelity (LiFi), a Worldwide Interoperability for Microware Access (WiMax), an ad hoc network, an intranet, the Internet, a satellite network, a GPS network, a fiber optic-based network, and/or combination of these or other types of networks. Additionally, or alternatively, network 500 may include a cellular network, a public land mobile network (PLMN), a second generation (2G) network, a third generation (3G) network, a fourth generation (4G) network, a fifth generation (5G) network, and/or another network.

In embodiments, network 2501 may allow for devices describe any of the described figures to electronically communicate (e.g., using emails, electronic signals, URL links, web links, electronic bits, fiber optic signals, wireless signals, wired signals, etc.) with each other so as to send and receive various types of electronic communications.

User device 2502 and/or 2504 may include any computation or communications device that is capable of communicating with a network (e.g., network 2501). For example, user device 502 and/or user device 2504 may include a radiotelephone, a personal communications system (PCS) terminal (e.g., that may combine a cellular radiotelephone with data processing and data communications capabilities), a personal digital assistant (PDA) (e.g., that can include a radiotelephone, a pager, Internet/intranet access, etc.), a smart phone, a desktop computer, a laptop computer, a tablet computer, a camera, a personal gaming system, a television, a set top box, a digital video recorder (DVR), a digital audio recorder (DUR), a digital watch, a digital glass, or another type of computation or communications device.

User device 2502 and/or 2504 may receive and/or display content. The content may include objects, data, images, audio, video, text, files, and/or links to files accessible via one or more networks. Content may include a media stream, which may refer to a stream of content that includes video content (e.g., a video stream), audio content (e.g., an audio stream), and/or textual content (e.g., a textual stream). In embodiments, an electronic application may use an electronic graphical user interface to display content and/or information via user device 2502 and/or 2504. User device 2502 and/or 2504 may have a touch screen and/or a keyboard that allows a user to electronically interact with an electronic application. In embodiments, a user may swipe, press, or touch user device 2502 and/or 2504 in such a manner that one or more electronic actions will be initiated by user device 2502 and/or 2504 via an electronic application. User device 2502 and/or 2504 may receive electronic information from antenna 506 and generate and display graphs such as those described in the figures above.

User device 2502 and/or 2504 may include a variety of applications, such as, for example, an e-mail application, a telephone application, a camera application, a video application, a multi-media application, a music player application, a visual voice mail application, a contacts application, a data organizer application, a calendar application, an instant messaging application, a texting application, a web browsing application, a blogging application, and/or other types of applications (e.g., a word processing application, a spreadsheet application, etc.). In embodiments, user device 2502 and/or 2504 may be used to generate images associated with various types of eye diseases.

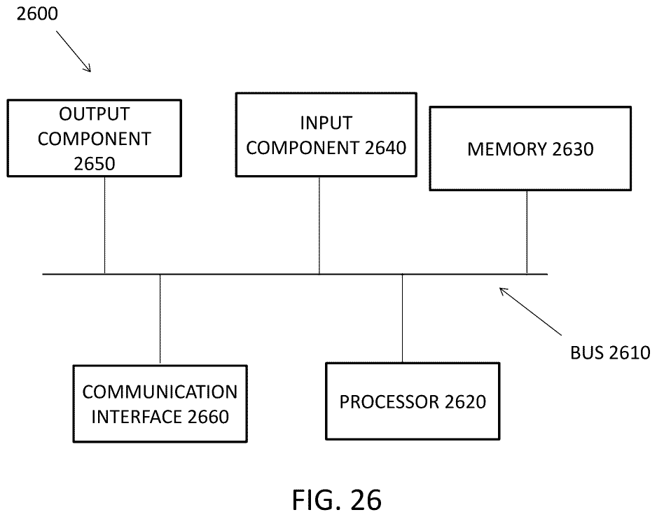

FIG. 26 is a diagram of example components of a device 2600. Device 2600 may correspond to user device 2502, or user device 2504. Alternatively, or additionally, user device 502 and user device 2504 may include one or more devices 600 and/or one or more components of device 2600.

As shown in FIG. 26, device 2600 may include a bus 2610, a processor 2620, a memory 2630, an input component 2640, an output component 2650, and a communications interface 2660. In other implementations, device 2600 may contain fewer components, additional components, different components, or differently arranged components than depicted in FIG. 26. Additionally, or alternatively, one or more components of device 2600 may perform one or more tasks described as being performed by one or more other components of device 2600.

Bus 2610 may include a path that permits communications among the components of device 2600. Processor 2620 may include one or more processors, microprocessors, or processing logic (e.g., a field programmable gate array (FPGA) or an application specific integrated circuit (ASIC)) that interprets and executes instructions. Memory 2630 may include any type of dynamic storage device that stores information and instructions, for execution by processor 2620, and/or any type of non-volatile storage device that stores information for use by processor 2620. Input component 2640 may include a mechanism that permits a user to input information to device 2600, such as a keyboard, a keypad, a button, a switch, voice command, etc. Output component 2650 may include a mechanism that outputs information to the user, such as a display, a speaker, one or more light emitting diodes (LEDs), etc.

Communications interface 2660 may include any transceiver-like mechanism that enables device 2600 to communicate with other devices and/or systems. For example, communications interface 2660 may include an Ethernet interface, an optical interface, a coaxial interface, a wireless interface, or the like.

In another implementation, communications interface 2660 may include, for example, a transmitter that may convert baseband signals from processor 2620 to radio frequency (RF) signals and/or a receiver that may convert RF signals to baseband signals. Alternatively, communications interface 660 may include a transceiver to perform functions of both a transmitter and a receiver of wireless communications (e.g., radio frequency, infrared, visual optics, etc.), wired communications (e.g., conductive wire, twisted pair cable, coaxial cable, transmission line, fiber optic cable, waveguide, etc.), or a combination of wireless and wired communications.

Communications interface 660 may connect to an antenna assembly (not shown in FIG. 6) for transmission and/or reception of the RF signals. The antenna assembly may include one or more antennas to transmit and/or receive RF signals over the air. The antenna assembly may, for example, receive RF signals from communications interface 660 and transmit the RF signals over the air, and receive RF signals over the air and provide the RF signals to communications interface 660. In one implementation, for example, communications interface 660 may communicate with network 501.

As will be described in detail below, device 2600 may perform certain operations. Device 600 may perform these operations in response to processor 2620 executing software instructions (e.g., computer program(s)) contained in a computer-readable medium, such as memory 2630, a secondary storage device (e.g., hard disk, CD-ROM, etc.), or other forms of RAM or ROM. A computer-readable medium may be defined as a non-transitory memory device. A memory device may include space within a single physical memory device or spread across multiple physical memory devices. The software instructions may be read into memory 2630 from another computer-readable medium or from another device. The software instructions contained in memory 2630 may cause processor 2620 to perform processes described herein. Alternatively, hardwired circuitry may be used in place of or in combination with software instructions to implement processes described herein. Thus, implementations described herein are not limited to any specific combination of hardware circuitry and software.

It will be apparent that example aspects, as described above, may be implemented in many different forms of software, firmware, and hardware in the implementations illustrated in the figures. The actual software code or specialized control hardware used to implement these aspects should not be construed as limiting. Thus, the operation and behavior of the aspects were described without reference to the specific software code—it being understood that software and control hardware could be designed to implement the aspects based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While various actions are described as selecting, displaying, transferring, sending, receiving, generating, notifying, and storing, it will be understood that these example actions are occurring within an electronic computing and/or electronic networking environment and may require one or more computing devices, as described in FIG. 19, to complete such actions.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

In the preceding specification, various preferred embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. A method, comprising:
receiving, by a user device, an electronic fundus scan; and
determining, by the user device, retinal disease information based on receiving the electronic fundus scan.

2. The method of claim 1, wherein the determining the retinal disease information includes analyzing by a convolutional transformer system,
wherein the convolutional transformer system includes:
an encoder,
a transformer, and
a decoder;
and the determining the retinal disease information further includes:
decomposing the electronic fundus scan into non-overlapping electronic patches.

3. The method of claim 2, wherein the encoder includes five levels.

4. The method of claim 2, further comprises:
generating an electronic report.
